(19) United States
(12) Patent Application Publication
(10) Pub. No.: US 2026/0157820 A1
(43) Pub. Date: Jun. 11, 2026

(54) ERGONOMIC SURGEON CONSOLE

(71) Applicant: 5by5 Medical LLC, Chicago, IL (US)

(72) Inventors: Andreas Vogler, Munich (DE); Lorenzo Giulianotti, Chicago, IL (US)

(21) Appl. No.: 18/972,167

(22) Filed: Dec. 6, 2024

(51) Int. Cl. A61B 90/60; A61B 34/00; A61B 34/35

(52) U.S. Cl. CPC A61B 90/60; A61B 34/25; A61B 34/35; A61B 34/74

(57) ABSTRACT

An ergonomic console configured for interfacing with a remote robotic surgical system is provided. The console includes a chassis, an ergonomic seat configured to receive the body of a surgeon, a main display to view the remote surgical field, an interface unit provided with control devices of the remote robotic surgical system configured to be operated by the surgeon and a core unit configured to remotely operate the surgical robotic system based on the operation of said control devices.

The console includes a plurality of moving units manual or automatically actionable, configured to allow the tilting and/or displacing of said seat, interface unit and main display with respect to said chassis according to user preferences.

### TECHNICAL FIELD

The present disclosure generally relates to surgery robot controlling systems and, in particular, to an ergonomic console configured for interfacing with a remote robotic surgical system by means of a cable or wireless connection.

### BACKGROUND

Nowadays, top surgeons worldwide contribute to the success of clinical robotic surgery, as robotic systems get more and more advanced. However, the ergonomic environment for these highly educated and trained specialists is alarmingly behind.

First, the consoles already available for controlling surgical robotic systems are usually meant to stay in predetermined position of the operation room. They could have wheels for mobility inside the room but, however, permitted movements are limited. In any case, the consoles are configured to be moved manually, under the push of an operator.

Such consoles offer limited possibility of ergonomic control to the surgeons, for example just by adjusting the hight of the monitor to match his/her preferences.

The surgeons are usually seated on uncomfortable stools or chairs, while working on robotic manipulators and assuming a curved position to lean towards a monitor, showing in real time the operations executed by the robotic system on the patient. Usually, the surgeon inserts his/her head within a seat housing a bifocal system, to which he/she must bring his eyes as close as possible to better see the surgical site images captured by the camera.

More particularly, the surgeon is bent forward with his/her hands stretched and eventually resting on a fixed board because the consoles don't provide lumbar support and tend to move backwards, moving the surgeon into an overbent or overstretched position at back and neck.

Usually, the robotic manipulators are placed in a fixed position, with no possibility for the surgeon to adjust their arrangement during the execution of the robotic surgery, to improve his/her comfort.

In other words, full customizing of the console configuration for matching the surgeon needs and preferences is not possible.

Also, there is no possibility to change the whole position/orientation of the surgeon itself when performing the required long-lasting, precision tasks.

This leads often to maintaining an uncomfortable position for the surgeon, for many hours at a time. Maintaining of an uncomfortable fixed position causes fatigue and reduction of proprioceptive capacity in the surgeon, eventually leading to a decrease in precision with the passing of the hours, thus negatively affecting performance.

Furthermore, a lot of physical recovery time is required for the surgeon between different operations.

In addition to the above drawbacks, the consoles in the state of the art are connected by cable only to the robotic surgical system associated thereto and offer no possibility to communicate with different devices.

In summary, consoles comprised in the state of the art are not more than (almost) static tele-manipulators, with limited customizability, usually intended to be arranged in a predetermined position of the operation room or to make small movements within the room, only if manually pushed by an operator.

### SUMMARY

The above drawbacks of the prior art solutions are overcome by a console for controlling a remote robotic surgical system constructed in accordance with the principles of the present disclosure, as defined in the independent claim 1.

Preferred features are the object of the examples set forth in the dependent claims.

A console according to the present disclosure is designed according to a user-centric philosophy, giving the user, i.e. the surgeon, the ability to fully customize the console settings according to his/her preferences. This imprinting is totally opposite to the already known static and robot-centric solutions.

More specifically, the console is provided with technical means suitable for adjusting the position and orientation of its components, to offer the user a positive experience while performing his/her tasks.

According to preferred examples, the proposed surgeon console realizes an ergonomic and integrated working environment, optimized for long-lasting high concentration work. The console allows placing the surgeon in a comfortable position, which can be deeply changed while performing the robot surgery tasks.

The console includes an ergonomic seat, configured to have adjustable position and orientation (i.e. inclination) in the workspace. Further, the console includes a control interface, provided with handling devices to control the activations of the remote surgical robot, such as one or more manipulators and/or pedals. The console can be configured to control not only the remote surgical instruments, but also other typology of electronic devices controlling the remote operation room environmental parameters, as well as patient's life support systems arranged in the operation room. Also, the manipulators and/or pedals enclosed in the console control interface can be configured to adjust their inclination and orientation in space according to the user preferences.

More in general, the console is provided with a plurality of moving units for adjusting the reciprocal seat and control interface position. There can be adjustable settings, pre-stored for example in the users'smartphone and therefore personalized, which can be transmitted to the console to customize it before use.

Also, the ergonomic seat can house devices for massaging the surgeon, effective for reducing tiredness and muscle tension, especially when performing long time operations (e.g. 7-8 hours). The console can further house support devices for the arms, to relax the muscles, for example in the form of an exoskeleton. Same solution can be provided for legs support.

Therefore, the use of the console according to the present disclosure allows to reduce the surgeon rest time between different interventions, allowing an improvement in the surgical interventions performing rate daily.

The result is that the surgeon can more comfortably operate patients located far away from him/her, even in a different continent, allowing a wider range of people to benefit from the medical services concentrated in the hands of a few specialists around the world.

Also, the proposed console could allow the expert surgeon to support other surgeons in the same or nearby operation rooms, as well as to support other remote interventions. Advantageously, the console could be used for training purposes.

According to other preferred aspects of the present disclosure, the console is configured to provide an immersive or 3D vision of the surgical site, with the possibility for the surgeon of using manipulators like knobs to move the robotic arm and exploiting augmented reality (also, without knobs, but using touch screens or other kind of smart tactile interfaces). Such last opportunity can be further supported by the presence of a viewer in the form of glasses, which can be included in the console.

Therefore, the console according to the disclosure offers an integrated control of the operation room and events, and it is applicable for both on-site and remote surgery.

According to further examples, the console is provided with wheels, an electric motor and a respective battery, to implement a compact electric machine. This implementation allows to adapt the position of the console to the actual operating field. In addition, the surgeon on board the console can freely move to other operation rooms, even to assist another surgeon, increasing the efficiency of the interventions.

The console can also act autonomously and move within the hospital, for example following designated routes (ex. to collect blood bags), or even outside it.

The console can also comprise connections devices configured to access the Internet and hospital Intranet networks, which allows the surgeon to collect clinical data, images, etc., in a preparatory phase of the operation, or to edit the operation, for example for educational purposes.

In essence, the object of the present disclosure is no longer a static system, but a dynamic one. It improves the performance of the surgeon, and therefore the quality of the surgical operations and the safety of the patient. Obviously, the console can be configured to be interoperable with different robot systems that work on different patients.

The proposed robotic surgery console is a demonstration of the immense potential not only in developing robotic and AI-driven tools and machines for surgeons, but also in defining a new standard of working environment for these experts.

The proposed console can provide basically three main, integrated functions, that render it a solution provided with unique features:
1) best ergonomic sitting for telemanipulation;
2) electrical car/autonomous mini robot configuration;
3) powerful computer station for data processing (pre-and post-operative data).

The basic idea underlying the present disclosure is to create a professional ergonomic hi-concentration working environment, where highly skilled expert controls expensive machines.

Given this, the console comprises a dashboard which could work as a working space for all kinds of modern computer-implemented tasks: checking email, participating on meetings, writing scientific papers, and so on.

To this extent, a keyboard/mouse can be projected on or integrated in an armrest, or being flipped out from a console side portion.

Advantageously, the console could be used beyond robotic surgery environment, such as: civil/military drone control/aviation and civil/military robot control (de-mining, nuclear environment, catastrophe, sewage systems control, robotic submarines, space missions, et similia).

Other advantages, features and use modes of the object of the present disclosure will result evident from the following detailed description of some examples, not shown with limitative purposes.

### BRIEF DESCRIPTION OF FIGURES

In the following description, the enclosed Figures will be referred to, wherein:

FIGS. 1 and 2 respectively show a frontal and a lateral view of a preferred embodiment of a console according to the present disclosure;

FIG. 3 shows a perspective rear view of the console of FIG. 1;

FIG. 4 shows a perspective frontal view of the console of FIG. 1; and

FIG. 5 shows a scheme exemplifying another preferred embodiment of the console according to the present disclosure.

The above-mentioned Figures are to be meant exclusively by way of example and not for limitative purposes.

### DETAILED DESCRIPTION OF PREFERRED EXAMPLES

Several variants of the proposed console are set forth herein, although other examples are contemplated as well.

The different examples and variants can be used in combination, when compatible.

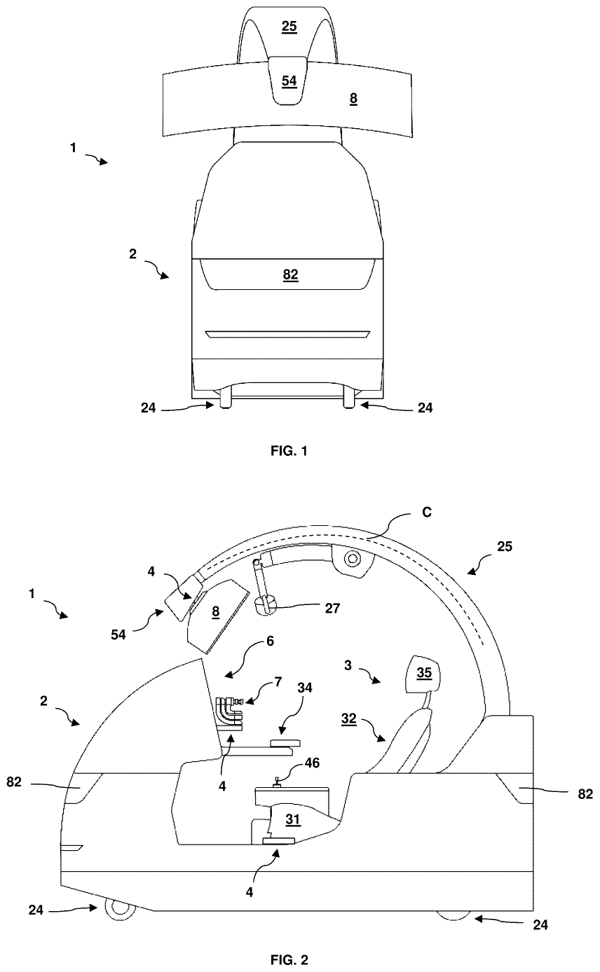

With reference to FIGS. 1, 2, 3 and 4, a console according to an exemplary embodiment of the present disclosure is set forth generally at 1.

The console 1 is conceived and configured to remotely control (by cable or wireless) a robotic surgical system 10 schematically shown in FIG. 5, therefore is intended to be used by a user like a surgeon or, more generally, by medical staff.

The console 1 comprises a chassis 2, which can be configured as a shell. The chassis 2 can comprise accessory systems (not shown in the Figures), including for example at least one of ventilation devices, heating/cooling devices, inner lights. Furthermore, on the external surface of the chassis 2, at least a service display 82 can be arranged, showing the name of the user (surgeon) who is onboard and/or specifications about the intervention in progress, and/or also configured to signal emergency situations.

The chassis 2 is preferably conformed as a standalone body, which can be configured not only to stand in a fixed position in an operation room, but also

automatically move towards other locations, as needed. To this extent, the console 1 can include wheels 24 arranged at a bottom part of the chassis 2. The wheels 24 are driven by an electric motor, included in the chassis 2 with its own rechargeable battery.

The chassis 2 comprises, or better includes within, a seat 3 configured to receive the body of the user, which is preferably an ergonomic seat. The access of the user to the seat 3 within the chassis 2 can be performed at a lateral side thereof, which can be eventually provided with a door (not shown in the Figures). Also, two doors can be provided, each arranged at a right and left lateral side of the chassis 2, respectively. The access door(s) can be locked by a closing device provided with a user recognition system, to prevent unauthorized users access to the inner of the chassis 2.

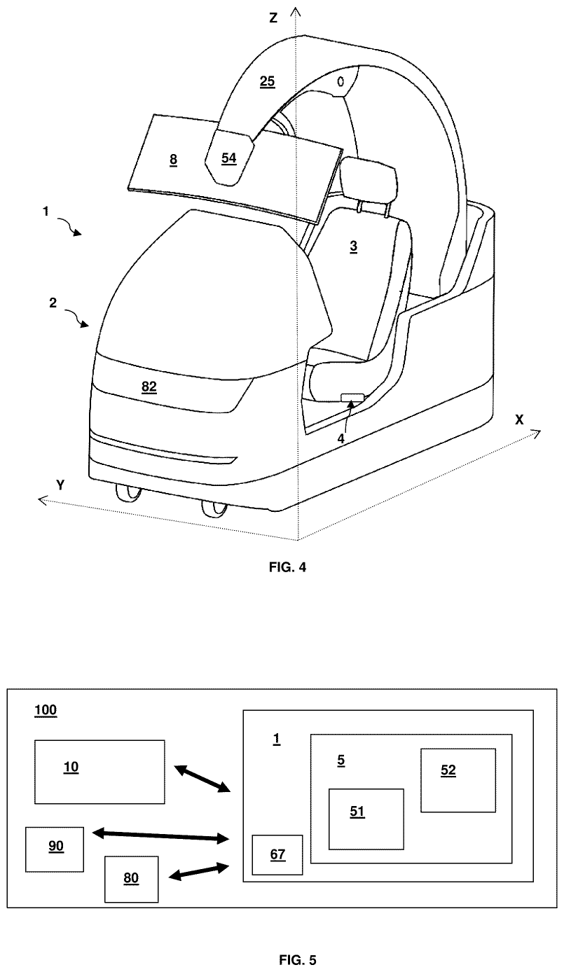

The seat 3 includes a base portion 31, a back portion 32 and preferably a headrest portion 35. The base portion 31 and back portion 32 can include massaging devices arranged therein. The seat 3 is configured to adjust its global spatial position and orientation with respect to chassis 2, thanks to the presence of respective one or more moving units 4. The degrees of freedom of the seat 3 with respect to the chassis 2 can be defined according to an exemplary 3D spatial reference system, such the Cartesian system shown in FIG. 4. Such degrees of freedom are at most six: translations and/or rotations according to each of the three axes X, Y, Z. According to a preferred implementation, the X axis extends according to a longitudinal direction coincident with the lateral size of the console 1, the Y axis extends according to a transversal direction coincident with the frontal size of the console 1, and the Z axis extends according to a sagittal direction coincident with the sagittal size of the console 1, that is its height.

The configuration of the console 1 can be such that the moving units 4 directly mechanically connect the seat 3 to the chassis 2, without interposition of further mechanical elements or components. According to an embodiment, the console 1 comprises a supporting platform preferably fixed at the base portion of the seat 3 or integrated therewith, wherein the moving unit(s) 4 is configured to connect such supporting platform to the chassis 2, allowing the moving of the seat 3 according up to six degrees of freedom.

According to non-limiting embodiments of the invention, one or each moving unit can include one or more of the following: rotary motors, linear motors, electric motors, electromagnetic motors, actuators, transmission components and/or equivalents thereof.

The tilting and/or displacing of the seat 3 allows the user to select the more effective and comfortable position to perform his/her tasks. The spatial position and orientation of the seat 3 can be adjusted at any time, even during the performance of the tasks. This helps to change pressure zones and support fluid shifts to avoid accumulating pain.

The seat 3 can further comprise one or more (two) armrests 34, each including one or more respective moving units 4 configured to allow the tilting and/or displacing thereof with respect to the base portion 31 and/or the back portion 32. Alternatively, the armrests 34 can be directly connected to chassis 2 by means of respective moving units 4, allowing tilting and/or displacement thereof with respect to the chassis 2, as already disclosed. Each armrest 34 can be in the form of an exoskeleton, configured to house, at least partly, a respective arm of the user and support its movements. Also, legs support elements, even conformed as exoskeletons, can be included in the console for supporting the legs of the user and connected to the chassis 2 by means of respective moving units 4.

Furthermore, each of the base portion 31, back portion 32 and headrest portion 35 can include its own moving unit 4, to adjust its spatial position and/or orientation: the base portion 31 with respect to the chassis 2 or the platform (if present), the back portion 32 with respect to the base portion 31 and the headrest portion 35 with respect to the back portion 32. At least two of the base portion 31, back portion 32 and headrest portion 35 can be integrated into a unitary seat element, even if keeping their own independent moving units 4 to allow customizing their orientation/position.

The console 1 according to the present disclosure comprises an interface unit 6 including one or more control devices 7, configured to be operated by the user for operating the remote robotic surgical system 10. Preferably, said control devices 7 are mainly arranged in front of the seat 3. The interface unit 6 can present the form of a unitary element, for example a panel, bearing all the control devices 7. According to such implementation, the panel can be fixed at the chassis 2 or integrated therewith. Alternatively, one or more respective moving units 4 can be interposed between the panel and the chassis 2, allowing the moving of the interface unit 6 according up to six degrees of freedom, as already described with reference to the seat 3.

According to another aspect of the disclosure, the control devices 7 can be each provided with its own moving unit 4, to adjust its position and/or orientation with respect the chassis 2 independently of each other.

The console 1 can be provided with one or more control devices 7 in the form of manipulators 75, preferably conformed as handlings. According to favourite aspects of the disclosure, the configuration of manipulators 75 is such that the remote robot instruments can be controlled by their movement, preferably in a manner that reproduces their movements. However, the control devices 7 included in the console 1 can be not only tools to be handled, like knobs, manipulators or joysticks, but also buttons or touch pads provided with differentiated activation areas, each associated to a different surgical instrument of the remote robotic system. As way of example, data glove systems could be included in the console 1, in replacement or addition to the robotic manipulators. In use, the surgeon holds light data tools, which are tracked by cameras located in an inner frontal portion of the chassis 2, using body tracking technology.

The control devices 7 can comprise foot pedals 76 too, which can be connected to the chassis 2 by means of respective moving units 4. Alternatively, all foot pedals 76 can be embraced by only one dedicated bottom platform, connected the chassis 2 by means of respective one or more moving units 4.

The delay between activation of the control devices 7 by the user and sending of the respective control signal to the remote robot system of course shall be compatible with the correctness and safety execution of the operation in the remote location.

In this regard, the console 1 is provided with connection means suitable for sending control signals to the remote robotic system configured to perform medical procedures, so that the local user of the console can operate on a patient located in a location remote therefrom.

Such connection means could be in the form of one or more cables. In alternative (or in addition), for implementing a wireless connection, wireless connection devices are comprised in a core unit 5, as will be better explained below.

As shown in FIG. 5, the core unit 5 is configured to remotely operate a surgical robotic system 10, based on the operation of the above-mentioned control devices 7. Therefore, the core unit 5 is configured to detect or receive operational data of the control devices 7, process them and send corresponding control commands to the remote robotic system, preferably in real time—except for the delay due to the actual time of transmission of the signal from the console 1 to the remote robotic system 10.

The console 1 is preferably configured to be connected to the remote surgical robotic system 10 by cable, to allow lower latency and data transmission security. Preferably, the connection with the remote surgical robotic system 10 is of ‘plug and play’ type.

The console 1 can be equipped with a power cable for connection to the operation room power supply. The power supply is able to recharge the console battery, that can be used as a power bank and/or to automatically move the console 1.

Preferably, the cable configured to connect the console 1 to the remote robotic surgical system 10 is both a data cable and a power cable, to power supply the remote robotic surgical system 10 in case of need.

The console 1 can also comprise a battery, even a different battery with respect to that associated to the electric motor, configured to power supply the remote robotic surgical system 10. The console and/or the robotic surgical system should always be connected to power supplies even when disconnected from the network, to deal with emergencies.

In other words, the console 1 can be wireless rechargeable, and/or include a battery rechargeable by an external cable or by a wireless charging system and configured to: automatically move and act as a backup and/or power bank also for the robotic surgical system 10 connected to it by cable, when power failures occur.

As already introduced, the core unit 5 can (also) be configured to wireless operate the surgical robotic system 10.

With particular reference to FIG. 3, the console 1 further includes a main display or monitor 8, arranged at an advanced position with respect to the seat 3 (i.e. facing the seat) and configured to receive and reproduce real time images, for example those acquired at the remote robotic surgical system, to shown to the user (surgeon) the surgical field, in real time. The main display can further be provided with audio devices to reproduce audio data, received for example from the remote robotic surgical system/operation room.

The main display 8 can have a curved surface, preferably extending as to circumscribe a space portion in front of the seat 3, thus allow an immersive view of the surgical field. The display 8 can be a 3D display.

To better cover the space in front and laterally to the seat 3, one or more secondary displays or monitors (not shown in the Figures) can be comprised in the console 1. The secondary displays can be arranged preferably at a lateral and/or lower position with respect to the main display 8. The images reproduced by the secondary displays can be different from those shown by the main display 8. Advantageously, while performing a remote surgical operation, the user can visualize the surgical field in real time and simultaneously view patient medical data on the different display(s).

Similar to the seat 3 and the interface unit 6, the main display 8 and the eventual secondary display(s) can be displaceable and/or tiltable to adjust their spatial positioning and orientation with respect to the chassis 2. Preferably, each display can be provided with its own moving unit 4, configured to allow up to six degrees of freedom, as already disclosed.

All the moving units 4 so far introduced can be manually or preferably automatically actionable, for example according to preferences listed in user profiles stored in the core unit 5, as explained in more detail below.

The core unit 5 can comprise a memory unit 51, wherein a user profile associated to each of the users authorized to access to the console 1 can be stored. Each user profile can include the respective list of access data, authorizations and restrictions, as well as preferred position/orientation data of the seat 3, the interface unit 6 (and/or each control device 7) and the main/secondary displays, with respect to the chassis 2. According to such embodiment, once the user accesses the console 1, the core unit 5 is programmed to automatically activate and control the moving units 4 to realize his/her favourite position/orientation configurations, according to the position/orientation data reported in the stored used profile. Such solution allows the user to enjoy his/her favourite console configuration without exercising any action, in a completely automatic and time-saving way.

The memory unit 51 can be further configured to store all the visual and operational data exchanged between the console 1 and the remote robotic system 10 during the intervention, starting from the user access to the console 1 until the leaving of the user.

In addition, the console 1 is provided with a dashboard 9, preferably included in the interface unit 6, configured to allow the user access to the core unit 5 and control all devices enclosed in the console 1 itself. The dashboard 9 can comprise a touch screen or an alphanumeric keyboard connected to a dedicated monitor.

The dashboard 9 could realize a working space for all kind of modern computer-implemented tasks: checking email, participating on meetings, writing scientific papers, and so on, thanks to Internet connection.

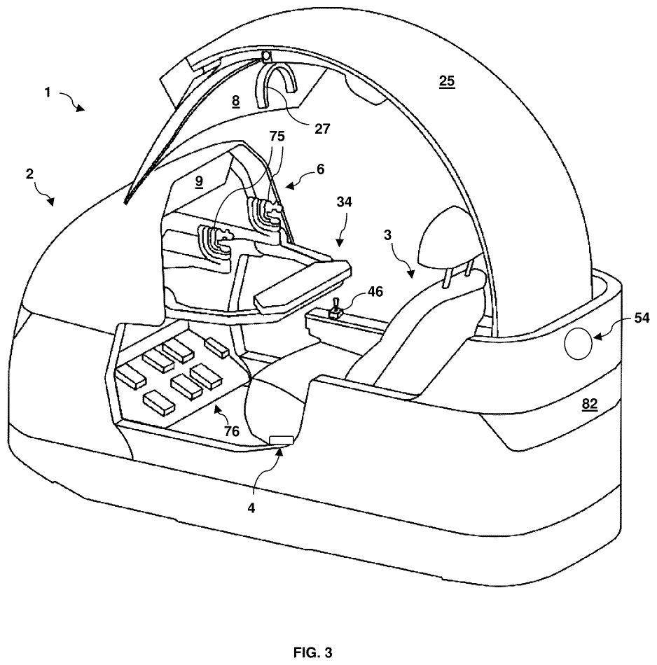

More particularly, the user can automatically control each of the moving devices 4 comprised in the console 1 by the dashboard 9. By means of the dashboard 9, the user can also control the eventual moving of the console 1, thanks to the already mentioned presence of the wheels 24 and the respective electric motor. Alternatively, a steering wheel and/or better a joystick (denoted by the number 46 in FIGS. 2 and 3) could be provided to control to moving of the console 1.

In this way, the user (surgeon) can move to an operation room to another on board the console 1.

The core unit 5 can be further programmed to support an automatic way of driving of the console 1. According to this embodiment, the console 1 can comprise a GPS system or at least a localization system. The console 1 is preferably further provided with a plurality of front, lateral and rear external cameras 54 connected to the core unit 5. The core unit 5 is configured to receive image data from the cameras 54 and process them to realize an autonomous driving of the console 1. Front and rear headlights and signal lights can be included in the console 1, as well as means of playing acoustic signals, which can be activated automatically by the core unit 5 when the console 1 is in motion.

In the memory unit 51, a plurality of predetermined path and site of interests can be stored and selected by the user by using the dashboard 9.

The core unit 5 can be further configured to determine the automatic representation of the user's name, specification of the intervention in progress and/or report emergencies on the one or more service displays 82 arranged externally to the chassis 2.

According to a preferred variant, the core unit 5 can support a software for implementing a remote control of the console 1, for example for allowing the movement thereof, from one operation room to another, even without the user being onboard.

Furthermore, the core unit 5 can comprise a communication module 52, also connected to the dashboard 9, configured to realize a cable and/or wireless connection with the Internet network and/or an intranet network of a structure wherein the console is located. Advantageously, by using the dashboard 9, the user with appropriate authorization level can access to the hospital servers and databases, to extract data relative to the patient even during the performing of the remote surgical operation on board the console 1. The data can include exams results, pre-operative images, ECG, CT scans, medical history, etc., from medical records.

Such data can be preferably shown on the secondary displays, so that the user can enjoy simultaneous multiples views.

Furthermore, the communication module 52 can be configured to send/receive data from external electronic devices, such that a smartphone of the user, provided with a dedicated Application. This implementation allows the user to control the activation of moving devices 4 and accessory systems comprised in the chassis 2 by such external electronic devices. For example, the user can use the smartphone to send his/her preferences listed in the user profiles, allowing for simple and agile setup of the console.

The above-mentioned communication module 52 can be the same one responsible for sending control signals to the remote surgical robotic station 10.

According to an advantageous aspect of the present disclosure, the console 1 comprises an upper arm 25 configured to support the main display 8 at a frontal position with respect to the seat 3. The upper arm 25 preferably extends from a back side of the chassis 2, according to a curved development direction C, as shown in FIG. 2. The upper arm 25 can be fixed with respect to chassis 2 or be realized as an integral part thereof. Preferably, the upper arm 25 is C-shaped, with concavity facing the seat 3. Even more preferably, the upper arm 25 has a transversal extension (width, according to the Y axis) large enough to cover the entire size of the seat 3, seen from above (i.e. top view, according to Z axis).

The upper arm 25 has a free terminal end at which the main display 8 is connected, arranged in front of the seat 3. Preferably the main display 8 is connected to the upper arm 25 by interposition of a respective moving unit 4.

According to other examples of the disclosure, the console 1 comprises a pair of augmented reality/virtual reality glasses or viewer 67 (schematically shown in FIG. 5), that can be able to realize an immersive (better 3D) vision of the surgical site. The viewer 67 can be configured to magnify images of the surgical site. Such augmented reality viewer 67 can be connected to the upper arm 25 by means of a cable. The console 1 can also comprise a viewer seat 27 for housing it, when not in use by the user. The viewer seat 27 can be provided in a more rearward position on the upper arm 25, with respect to the main display 8.

The main display 8 as well as the augmented reality glasses 67 can be additionally utilized for supporting the user when the dashboard 9 is used for performing computer-implemented tasks other than performing of a remote surgical operation, e.g. checking email, participating on meetings, writing scientific papers.

According to the present disclosure, an operation room 100 comprising a remote robotic surgical system 10 and a console 1 according to what already described is also provided, as shown in FIG. 5. It is noted that the console 1 can be located in any position inside/outside of the operation room 100.

The operation room 100 can comprise electric/mechanical devices 90 configured to adjust its environmental parameters, such that air composition, air flow, lighting and/or temperature inside it, and/or to drive automatic access doors. Advantageously, the console 1, in particular the core unit 5 provided with the communication module 52, can be configured to control the activation of said electric/mechanical devices 90. In other words, the console 1 allows to remotely control the accesses and/or adjust the environmental parameters of the operation room 100. Furthermore, the console 1 can be configured to remotely operate and control all the patient's life support systems 80 arranged in the operation room 100, such as anaesthesia machine, ECG control, blood pressure, etc. In essence, all the patient life support systems, which can be integrated or not on the surgical robot (i.e. integrated in the operation room), could be remotely controlled by the proposed console 1. Such control could be realized by means of a wireless connection, or according to a wired connection.

Specific examples of implementation of the present disclosure are further provided below.

### Example 1

A console 1 configured to remotely control a robotic surgical system 10, comprising:
a chassis 2;
an ergonomic seat 3 configured to receive the body of a user, provided with a base portion 31 and back portion 32;
a main display 8 configured to receive and reproduce real time images;
an interface unit 6 including one or more control devices 7 configured to be operated by the user;
a core unit 5 configured to remotely operate the surgical robotic system 10 based on the operation of said control devices 7; and
augmented reality viewer 67 for immersive vision of the surgical field,
wherein said console 1 further comprises:
a plurality of moving units 4, respectively connecting said seat 3, interface unit 7 and main display 8 to said chassis 2, and configured to allow the tilting and/or displacing of said seat 3, interface unit 6 and main display 8 with respect to said chassis 2, independently form each other.

### Example 2

The console 1 according to example 1, comprising an upper arm 25 configured to support said main display 8, said upper arm 25 extending from a back side of said chassis 2 according to a curved development direction C, preferably C-shaped with concavity facing said seat 3.

### Example 3

The console 1 according to example 2, wherein said upper arm 25 has a free terminal end at which said main display 8 is connected, preferably by means of a respective moving unit 4.

### Example 4

The console 1 according to examples 2 or 3, wherein said upper arm 25 has a width large enough to cover the entire size of said seat 3, seen from above.

### Example 5

The console 1 according to any of examples 2 to 4, wherein said upper arm 25 comprises a viewer seat 27 configured to house said augmented reality viewer for immersive vision of the surgical field.

### Example 6

The console 1 according to any of the preceding examples 1 to 5, comprising one or more armrests 34, respectively comprising one or more moving units 4 configured to allow the tilting and/or displacing of said armrests 34 with respect to said chassis 2.

### Example 7

The console 1 according to the preceding example 6, wherein each of said armrests 34 has an exoskeleton configuration, being configured to house a respective arm of the user and support its movements.

### Example 8

The console 1 according to any of the preceding examples 1 to 7, wherein said control devices 7 comprise manipulators 75 and/or foot pedals 76, at least one of them being connected to said chassis 2 by means of a respective moving unit 4.

### Example 9

The console 1 according to any of the preceding examples 1 to 8, wherein said core unit 5 comprises a communication module 52 configured to realize a cable and/or wireless connection with an Internet and/or Intranet network.

### Example 10

The console 1 according to any of the preceding examples 1 to 9, comprising one or more secondary displays arranged at a lateral and/or lower position with respect to said main display 8.

### Example 11

The console 1 according to any of the preceding examples 1 to 10, comprising wheels 24 arranged at a bottom portion of said chassis 2 and driven by an electric motor, for allowing movement of said console 1.

### Example 12

The console 1 according to the preceding examples 1 to 11, comprising one or more external cameras 54, wherein said core unit 5 is configured to receive image data from said cameras 54 and process them to realize an autonomous driving of said console 1.

### Example 13

Use of the console according to the preceding examples 1 to 12 for the training of students, surgeons and/or medical staff.

### Example 14

Surgical system assembly comprising a robotic surgical system 10 and a console 1 according to any of the preceding examples 1 to 13, wherein said console 1 is configured to remotely control a robotic surgical system by means of a cable or wireless connection.

### Example 15

Operation room 100 comprising a robotic surgical system 10 and a console 1 according to any of the preceding examples 1 to 13, said operation room 100 comprising electric/mechanical devices 90 configured to adjust air composition, air flow, lighting and/or temperature inside it and/or to drive automatic access doors, wherein said console 1 is configured to remotely control said electric/mechanical devices 90 and/or patient's life support systems 80 arranged in said operation room 100.

It is understood that the foregoing detailed description is merely illustrative and is not to be taken as a limitation of the scope of an embodiment of the present disclosure, which is defined solely by the appended claims. Various changes and modifications to the disclosed examples will be apparent to those skilled in the art.

1. A console configured to remotely control a robotic surgical system, comprising:
a chassis;
an ergonomic seat configured to receive the body of a user, provided with a base portion and back portion;
a main display configured to receive and reproduce real time images;
an interface unit including one or more control devices configured to be operated by the user;
a core unit configured to remotely operate the surgical robotic system based on the operation of said control devices; and
an augmented reality viewer for immersive vision of the surgical field,
wherein said console further comprises:
a plurality of moving units, respectively connecting said seat, interface unit and main display to said chassis, and configured to allow the tilting and/or displacing of said seat, interface unit and main display with respect to said chassis, independently from each other.

2. The console according to claim 1, comprising an upper arm configured to support said main display, said upper arm extending from a back side of said chassis according to a curved development direction.

3. The console according to claim 2, wherein the curved development direction is C-shaped with concavity facing said seat.

4. The console according to claim 2, wherein said upper arm has a free terminal end at which said main display is connected.

5. The console according to claim 4, wherein the main display is connected to the free terminal end of the upper arm by a respective one of the plurality of moving units.

6. The console according to claim 2, wherein said upper arm has a width large enough to cover the entire size of said seat, seen from above.

7. The console according to claim 2, wherein said upper arm comprises a viewer seat configured to house said augmented reality viewer.

8. The console according to claim 1, comprising one or more armrests, respectively comprising one or more moving units configured to allow the tilting or displacing of said armrests with respect to said chassis.

9. The console according to claim 8, wherein each of said armrests has an exoskeleton configuration, being configured to house a respective arm of the user and support its movements.

10. The console according to claim 1, wherein said control devices comprise manipulators and/or foot pedals, at least one of them being connected to said chassis by a respective moving unit of the plurality of moving units.

11. The console according to claim 1, wherein said core unit comprises a communication module configured to realize a cable and/or wireless connection with an Internet and/or Intranet network.

12. The console according to claim 1, comprising one or more secondary displays arranged at a lateral and/or lower position with respect to said main display.

13. The console according to claim 1, comprising wheels arranged at a bottom portion of said chassis and driven by an electric motor, for allowing movement of said console.

14. The console according to claim 1, comprising one or more external cameras, wherein said core unit is configured to receive image data from said cameras and process them to realize an autonomous driving of said console.

15. A method of training students, surgeons or medical staff using the console of claim 1.

16. A surgical system assembly comprising a robotic surgical system and a console according to claim 1, the console being configured to remotely control a robotic surgical system by a cable or wireless connection.

17. An operation room comprising a robotic surgical system and a console according to claim 1, said operation room comprising electric or mechanical devices configured to adjust air composition, air flow, lighting and/or temperature inside it and/or to drive automatic access doors, wherein said console is configured to remotely control said electric or mechanical devices and/or patient's life support systems arranged in said operation room.
